(19) United States
(12) Patent Application Publication
(10) Pub. No.: US 2009/0095096 A1
(43) Pub. Date: Apr. 16, 2009

(54) Hazardous materials sensing robot

(76) Inventors: Charles Edwin Dean, Shrewsbury, MA (US); Jennifer R. Sarkis, Chelmsford, MA (US); Kurt Bruck, Providence, RI (US); John Young, Sutton, MA (US); Hoi-Yin Tong, Newton, MA (US); Christopher J. Langford, Wakefield, MA (US)

Correspondence Address: Iandiorio & Teska, 260 Bear Hill Road, Waltham, MA, 02451-1018, US

(21) Appl. No.: 11/973,652

(22) Filed: Oct. 10, 2007

(51) Int. Cl. G01N 33/00; G01T 1/00; H05G 2/00; H05G 1/26

(52) U.S. Cl. 73/866; 378/1; 378/193; 378/91; 901/1; 901/46

(57) ABSTRACT

A hazardous materials sensing robot includes a robot platform and a universal mounting tray on the robot platform for removably mounting thereon a plurality of sensors each having an output. An electronic interface unit is configured to receive the outputs of the sensors. An operator control unit remotely operates the robot platform. There is a communication link between the robot platform and the operator control unit for transmitting the sensor outputs to the operator control unit.

### FIELD OF THE INVENTION

This subject invention relates to robots used to detect and analyze hazardous or potentially hazardous materials and environments.

### BACKGROUND OF THE INVENTION

It is known to equip a remotely controlled robot with sensors and maneuver the robot to a location to detect the presence of different substances.

The typical user of such a system includes members of a police or SWAT team, environmental protection personnel, fire departments, and explosive ordinance disposal teams. Some users may not have sufficient training in electronics to properly configure a given sensor and operate it in conjunction with the robot.

Also, different types of sensors from different vendors are configured differently and produce outputs according to different protocols. There is a need for a system which can readily accept the outputs of different types of sensors. For example, on one mission, a team may require only a chemical sensor. On another mission, however, the team may require both a chemical sensor and an explosives detector. On still another mission, a gas detector may be required or an item or package may need to be X-rayed. In addition, additional equipment may need to be carried by the robot.

Finally, most robot platforms are fairly expensive. There may be instances where a given robot is used for missions where hazmat sensors are not required. In such a case, permanently mounted sensors may interfere with the mission and/or could be damaged.

### SUMMARY OF THE INVENTION

It is therefore an object of this invention to provide a hazardous materials sensing robot.

It is a further object of this invention to provide such a robot which is versatile and adaptable.

It is a further object of this invention to provide such a robot which readily accepts the outputs from different types of sensors and from sensors provided by different vendors.

It is a further object of this invention to provide such a robot which is easy to configure, reconfigure, and operate.

It is a further object of this invention to provide such a robot wherein the hazmat sensors and associated electronics and mounting hardware to be quickly removed so the robot can be used in non-hazmat situations and missions.

It is a further object of this invention to provide such a robot wherein the hazmat sensors and electronics to be quickly installed so the robot can be used in hazmat missions.

The subject invention results from the realization that by equipping a robot platform with a quick release universal mounting tray itself including an electronic interface configured to receive and process the outputs from different sensors, the hazmat robot of the subject invention, in one preferred embodiment, is highly versatile and adaptable and is easy to configure, reconfigure, and operate. In one configuration, the hazmat robot can also be equipped with an X-ray source.

The subject invention, however, in other embodiments, need not achieve all these objectives and the claims hereof should not be limited to structures or methods capable of achieving these objectives.

The subject invention features a hazardous materials sensing robot comprising a robot platform, a universal mounting tray on the robot platform for removably mounting thereon a plurality of sensors each having an output, and an electronic interface unit configured to receive the outputs of the sensors. An operator control unit remotely operates the robot platform. A communication link between the robot platform and the operator control unit is for transmitting the sensor outputs to the operator control unit.

The typical universal mounting tray includes quick release mounts facilitating removal of the universal mounting tray from the robot platform. An interface bracket is preferably provided for each quick release mount.

Further included may be a plurality of sensor brackets for mounting the sensors on the universal mounting tray. The typical universal mounting tray includes a broad flat top surface and the electronic interface unit is mounted on one end of the broad flat top surface. There may be a sensor mounting tray over the electronic interface unit.

Preferably, the electronic interface unit includes one or more processors and software which implements Joint Architecture Unmanned Systems operating thereon for accepting the outputs of a variety of different sensors. The system may include sensors for mounting on the universal mounting tray including a chemical sensor, a radiation monitor, a gas detector, an explosives detector, and/or a temperature probe. Preferably, a robot camera is aimable at the universal mounting tray for viewing the output of any additional sensor(s) mounted thereon not connected to the electronic interface unit. The system may also include a personal data assistant connectable to the operator control unit for displaying and logging the output of the sensors.

There may also be a mount on the universal mounting tray for an X-ray source. The electronic interface unit includes a connection for the X-ray source. The preferred mount is configured to pitch up and down via a motor. The motor is typically controlled by the operator control unit. Also included may be a laser mount for aiming the X-ray source.

The typical robot platform further includes an articulating arm including a distal X-ray film or imager holder. One holder includes a bracket pivotably attached to a rod. The operator control unit may include a touch screen display for controlling the X-ray source.

One preferred system in accordance with this invention includes a universal mounting tray removably securable to a robotic platform via quick release mounts and an electronic interface unit configured to receive the outputs of sensors mounted on the universal mounting tray. Also included may be a set of an interface bracket for each quick release mount. A plurality of sensor brackets can be added for mounting the sensors on the universal mounting tray. The typical universal mounting tray includes a broad flat top surface and the electronic interface unit is mounted on one end of the broad flat top surface. A sensor mounting tray can be positioned over the electronic interface unit.

The preferred electronic interface unit includes one or more processors and Joint Architecture Unmanned Systems software operating thereon for accepting the outputs of a variety of different sensors. The system may include sensors mounted on the universal mounting tray such as a chemical sensor, a radiation monitor, a gas detector, an explosives detector, and/or a temperature probe. Also, the universal mounting tray may include a camera mount for viewing the output of any sensor mounted thereon not connected to the electronic interface unit. The system may also include a personal data assistant for displaying and logging the output of the sensors.

For imaging parcels or other items, a mount on the universal mounting tray is typically configured for an X-ray source. The electronic interface unit includes a connection for the X-ray source. The preferred mount is configured to pitch up and down and is typically motor driven. The system may include a laser mount for aiming the X-ray source. One holder includes a bracket pivotably attached to a rod.

One hazardous materials sensing robot in accordance with this invention includes a robot platform including an articulating arm with a distal X-ray film holder and a mount for an X-ray source for emitting X-rays in the direction of the holder.

Preferably, a universal mounting tray is releasably mounted on the robot platform and the X-ray source mount is located on the universal mounting tray. And, an electronic interface unit is included on the universal mounting tray. The X-ray source is connected to the electronic interface unit. An operator control unit is for remotely operating the robot platform and the X-ray source.

A hazardous materials sensing system for a robot in accordance with this invention includes a universal mounting tray for mounting on a robot platform and for removably mounting thereon a plurality of sensors each having an output. An electronic interface unit on the universal mounting tray is configured to receive and process the outputs of the sensors. There may also be a tray over the electronic interface unit and another tray releasably mounted above the universal mounting tray.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram showing the primary components and subsystems associated with an example of a hazmat robot in accordance with the subject invention;

FIG. 2 is a schematic three-dimensional forward view of an example of a hazmat robot in accordance with the subject invention;

FIG. 3 is a schematic three-dimensional top view showing a sensor mounting tray and an electronic interface unit in accordance with the subject invention;

FIG. 4 is a schematic three-dimensional front view of the electronic interface unit shown in FIG. 3;

FIG. 5 is a schematic three-dimensional front view of an example of an operator control unit in accordance with the subject invention;

FIG. 6 is a schematic three-dimensional top view showing a personal data assistant or PDA interfaced with the operator control unit of FIG. 5;

FIG. 7 is a schematic front view showing the display of the PDA of FIG. 6;

FIG. 8 is a schematic three-dimensional top view of an example of a universal mounting tray in accordance with the subject invention;

FIG. 9 is another schematic three-dimensional view of the universal mounting tray shown in FIG. 8;

FIG. 10 is a schematic view of a robot in accordance with the subject invention now equipped with an X-ray source and X-ray film for imaging packages or objects;

FIG. 11 is a schematic three-dimensional side view showing an example where the X-ray unit of FIG. 10 is imaging an elevated package;

FIG. 12 is a schematic three-dimensional side view showing the X-ray source is now directing X-rays towards a package at a lower elevation;

FIG. 13 is a schematic closer view of an X-ray source and its mount;

FIG. 14 is a schematic three-dimensional top view showing a motor-driven tilting X-ray source mount in accordance with one preferred embodiment of the subject invention;

FIG. 15 is a schematic three-dimensional front view showing the connectors for an X-ray source and related equipment of the electronic interface unit;

FIG. 16 is a schematic view showing a touch screen on an operator control unit for controlling the X-ray source;

FIG. 17 is a schematic three-dimensional front view showing an additional top rack added to the universal sensor rack in accordance with the subject invention;

FIG. 18 is a schematic three-dimensional side view showing in more detail the primary components associated with the top tack of FIG. 17;

FIG. 19 is a schematic three-dimensional view showing the top rack of FIGS. 17 and 18 removed from the robot;

FIG. 20 is a block diagram showing the primary components of the operator control unit of FIG. 5; and

FIG. 21 is a block diagram showing the primary components of the electronic interface unit of FIG. 3.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

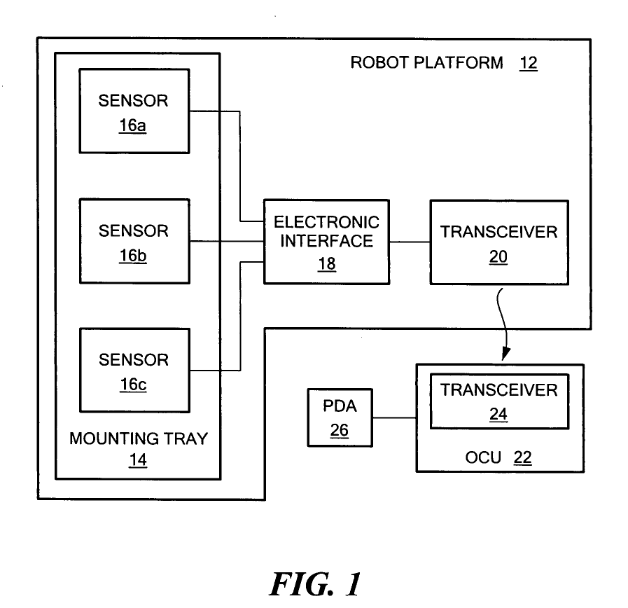

The subject invention, in one embodiment, includes robot platform 12, FIG. 1 and universal mounting tray 14 thereon for mounting a plurality of sensors 16a-16c. Electronic interface unit 18 receives and processes the outputs of each sensor. There is a communication link between robot 12 and operator control unit 22 whereby, via transceivers 20 and 24, the outputs of the sensors, as processed by interface unit 18, are transmitted to operator control unit 22 and, optionally, to personal data assistant or PDA 26.

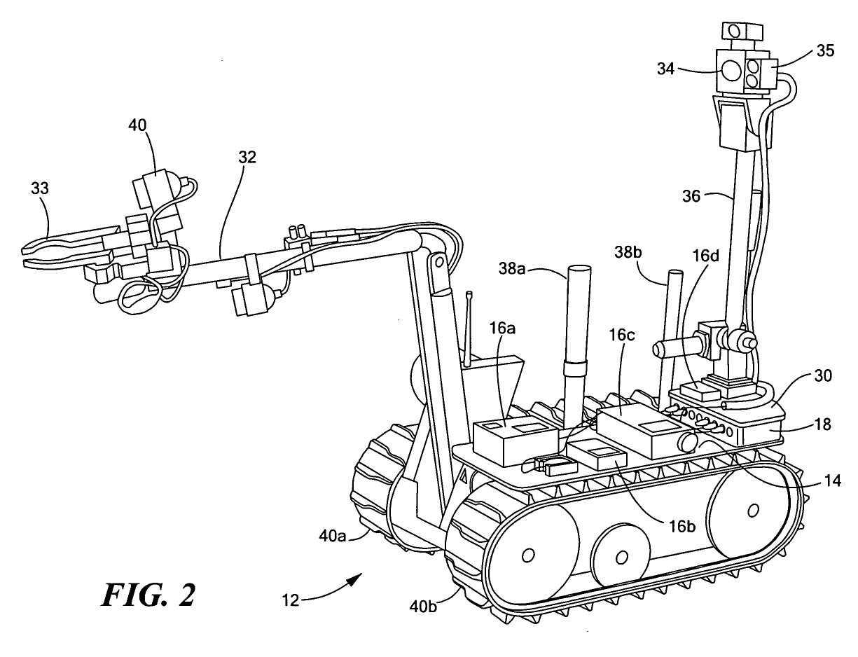

In one typical example, robot platform 12, FIG. 2 is a “Talon” brand robot (Foster-Miller, Inc. Waltham, Mass.). Other types of robot platforms, however, are possible. Universal tray 14 includes mounted thereon sensors 16a (e.g., an RAE multiRAE industrial gas sensor), 16b (e.g., a Can berra AN/VDR beta and gamma radiation detector), and 16c (a BAE systems “Chem Sentry 150 WMD” detector). Sensor 16d (a Raytek target temperature probe) is mounted on sensor mounting tray 30 positioned over electronic interface 18.

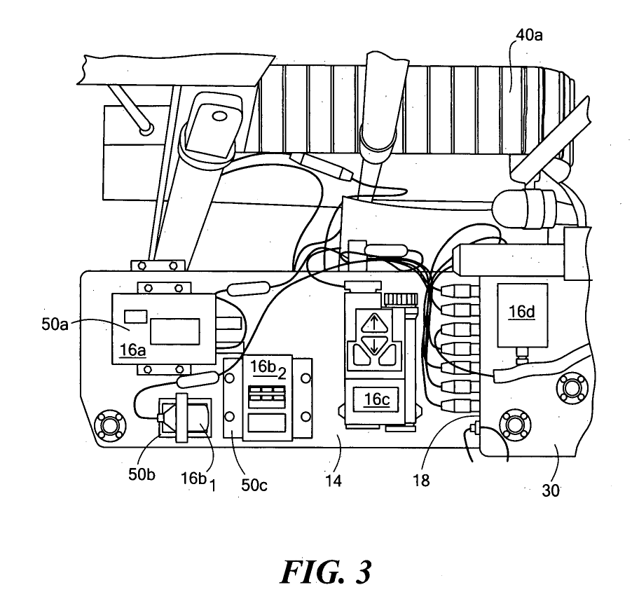

Robot 12 also includes articulating arm 32 with end effector 33 and camera 40. Camera 34 is on second robot arm 36. A laser and temperature probe 35 may also be included on robot arm 36. Antennas 38a and 38b are included for data and video transmissions between the operator control unit and the robot which is typically motor driven by side tracks 40a and 40b. FIG. 3 shows sensor brackets 50a, 50b, and 50c for sensors 16a, 16b1, 16b2, respectively.

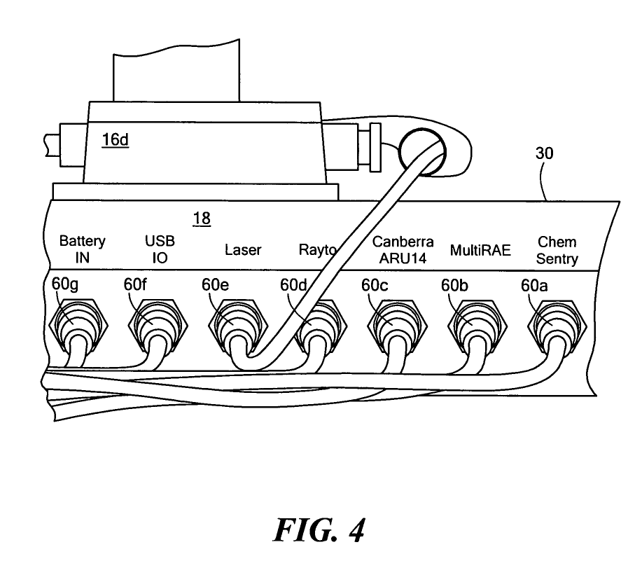

Cables connect each sensor 16a, 16b1, 16c, and 16d to labeled ports or connectors of electronic interface unit 18 also shown in FIG. 4. Connector 60a is for sensor 16c, connector 60b is for sensor 50a, connector 60c is for infrared sensor 16b1 serial device that reads sensor 16b2, connector 60d is for sensor 16d, and connector 60e is for laser and temperature probe 35, FIG. 2. Connector 60f is for a USB connection to the electronics of the robot platform and connector 60g is for battery power from the robot battery in order to power electronic interface unit 18 and sensors 16a and 50a. Other sensors can be used, however.

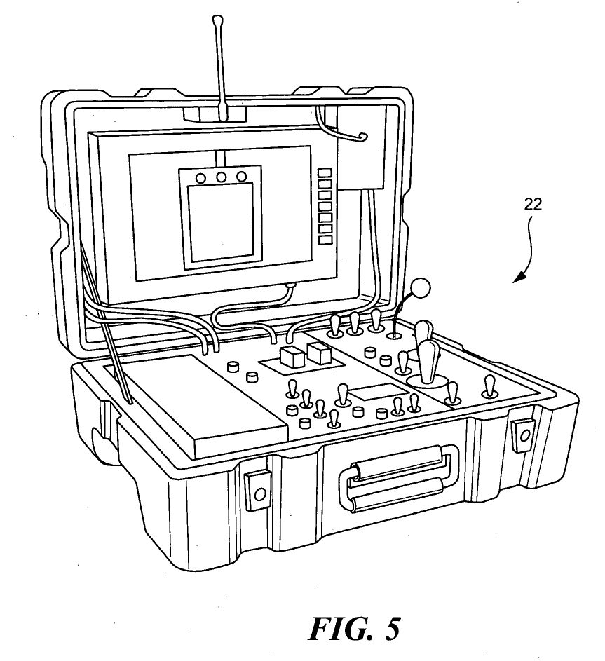

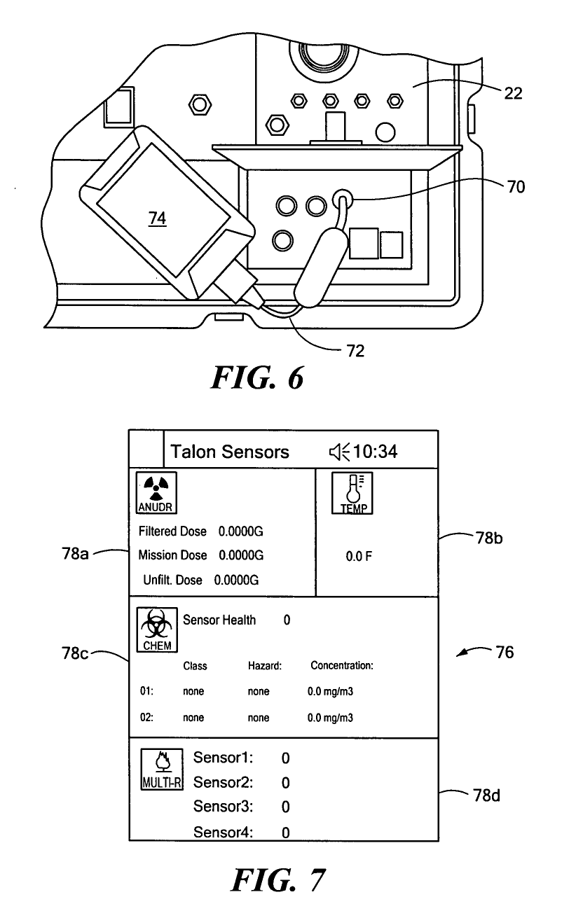

FIG. 5 shows operator control unit 22 which controls the movement of the robot and includes a transceiver therein which receives signals from the electronic interface unit typically via a transceiver on the robot. Operator control unit 22, FIG. 6 may also include Ethernet USB connector 70 which receives PDA cable 72 connected to optional PDA 74. The software of operator control unit 22 and electronic interface unit 18, FIGS. 1-4, allows the PDA to display and log the output of each sensor as shown in FIG. 7 where PDA display 76 includes display panels 78a-78d, one for each sensor as shown.

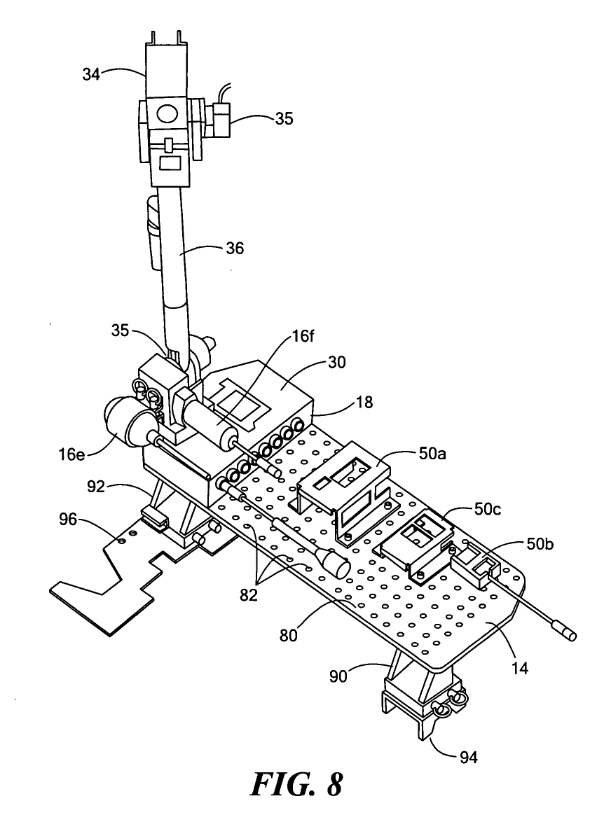

In this way, the user need only choose the sensors required for a particular mission, mount them on the mounting tray via their brackets, plug them into the interface, and read their outputs on the PDA display. Alternatively, the sensor outputs could be displayed on the operator control unit and/or on another remote computer or display. Preferably, electronic interface unit 18, FIG. 8 includes one or more processors running software that implements Joint Architecture Unmanned Systems (JAUS) for accepting the outputs of a variety of different sensors. See www.JAUS.wg.org.

FIG. 8 also shows in more detail universal mounting tray 14 typically made of aluminum. Tray 14 includes broad flat top surface 80 with an array of tapped holes 82 therethrough for mounting brackets 50a-50c and/or sensors thereto via fasteners. Electronic interface unit 18 is mounted on one end of top surface 80 as shown. Sensor mounting tray 30, on or over electronic interface unit 18, similarly includes tapped holes therethrough for mounting additional sensors and/or brackets. In FIG. 8, 16e is a camera and 16f is a motor housing for pan-tilt mast.

Universal mounting tray 14 preferably includes quick release mounts 90 and 92 releasably attached to robot interface interface brackets 94 and 96, respectively, secured to the robot platform. Brackets 94 and 96 may vary in design depending on the robot platform configuration.

In this way, hazmat tray 14 with the sensors and electronic interface unit thereon is easily removed from and then easily reattached to the robot. Camera 34 on pan-tilt mast 36 extending from mount 35 on tray 30 is aimable at tray 14 and/or tray 30 for viewing the output of any sensor not connected to electronic interface unit 18. In this way, if the software of electronic interface unit 18 does not support a particular sensor, or if there are not enough connectors for the number of sensors mounted on the trays 14 and/or 30, the readout of such sensors can still be viewed on operator control unit 22, FIG. 5 via camera 34.

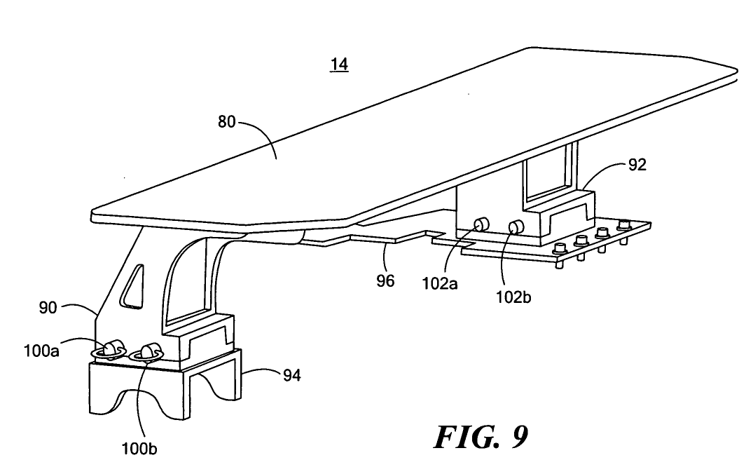

In one example, pins 100a and 100b, FIG. 9 releasably secure mount 90 to bracket 94 and pins 102a and 102b releasably secure mount 92 to bracket 96.

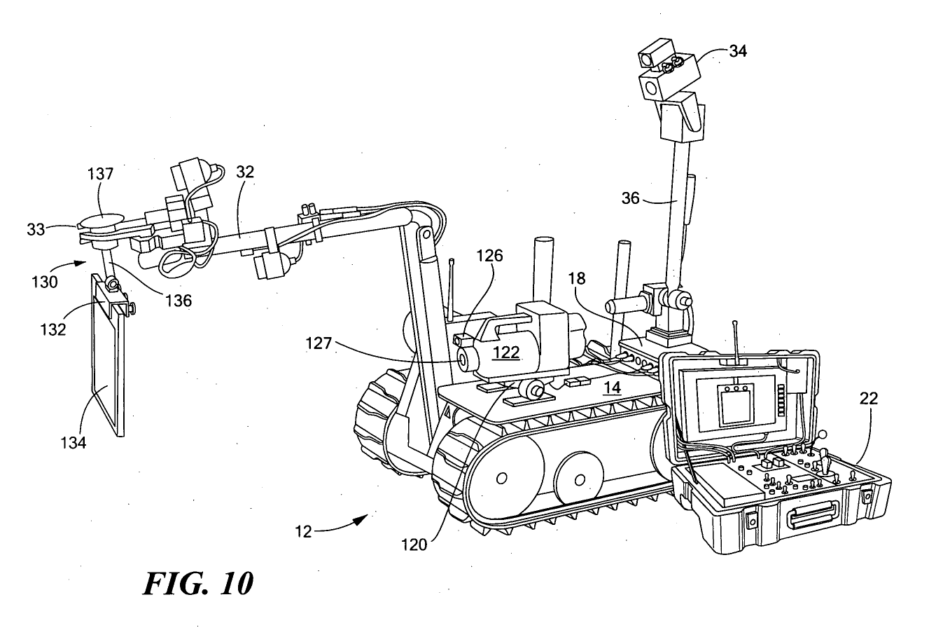

FIG. 10 shows an example where mount 120 is secured to tray 14 for X-ray source 122. Mount 120 is preferably motor driven to pitch up and down. Operator control unit 22 controls mount 120 and the X-ray source. Laser 126 on ring mount 127 can be added for aiming X-ray source 122. Articulating arm 32 of robot 12 includes distal X-ray film holder 130 which itself includes bracket 132 for film or an X-ray imaging system 134. Bracket 132 is hinged to rod 136 and free to pivot so film 134 remains perpendicular to the ground or surface. Rod 136 is attached to post 137 gripped by end effector 33.

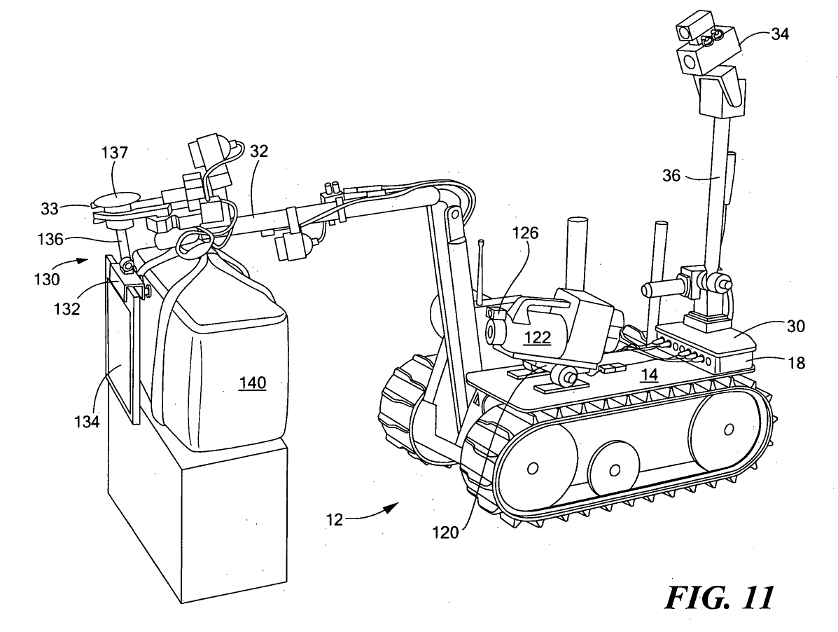

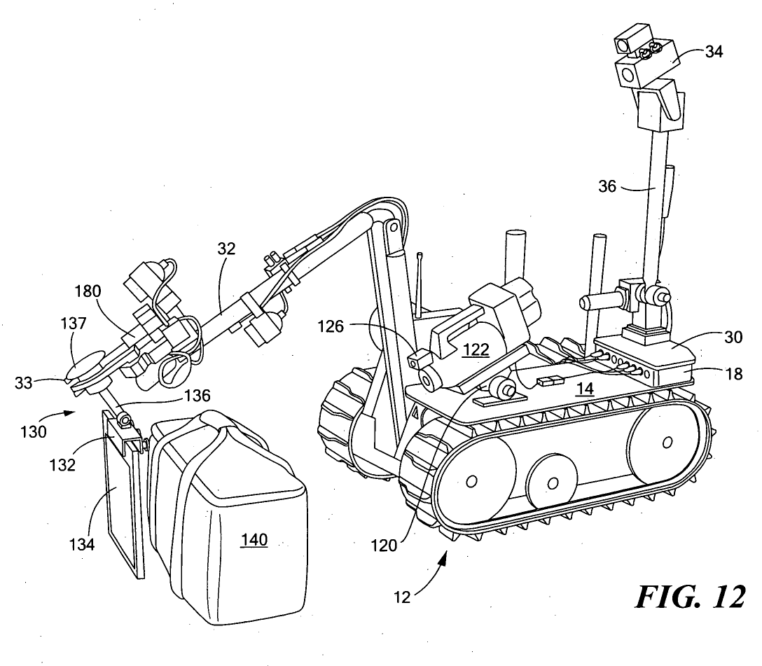

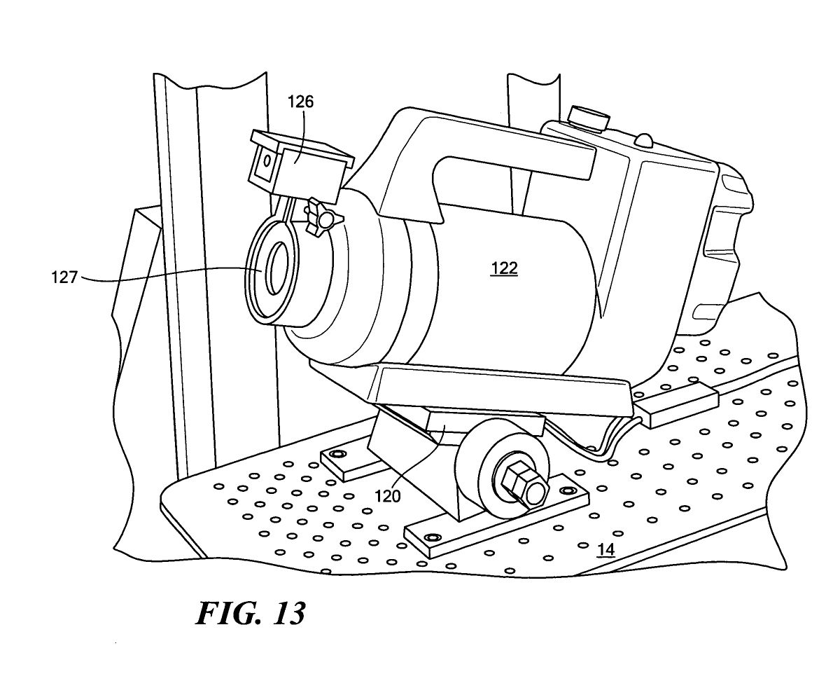

As shown in FIG. 11, robot 12 has been maneuvered proximate suspicious package 140, robot arm 32 has been moved to position X-ray film 134 behind package 140, and X-ray source 122 pitched upward via mount 120. In FIG. 12, X-ray source 122 is pitched downward via mount 120 for a package 140 located lower in elevation, for example on the same surface as robot 12. X-ray source 122, FIG. 13 may be an “XR200” available from Golden Engineering, Inc., Centerville, Ind. 47330. Camera 34 may be used to image package 140 and the output of laser source 126. Camera 34 can also be used to watch X-ray 122 display and pulse count.

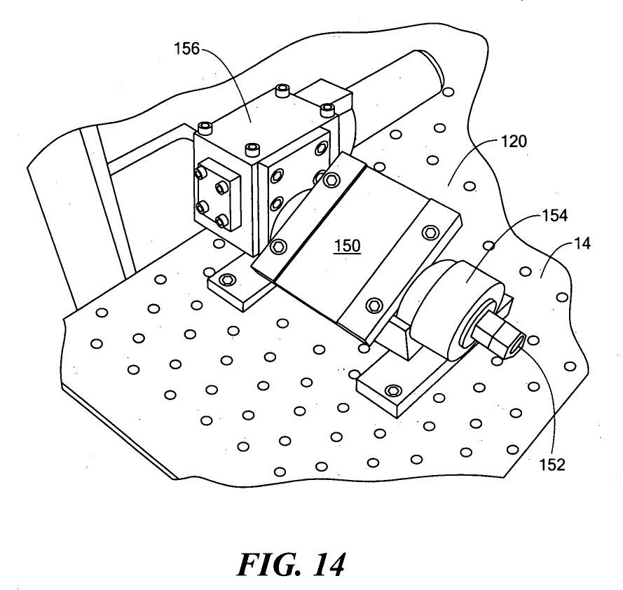

X-ray mount 120, FIG. 14 includes platform 150 rotatable via shaft 152 driven by motor 156. Friction system 154 allows mount 150 to be backdrivable.

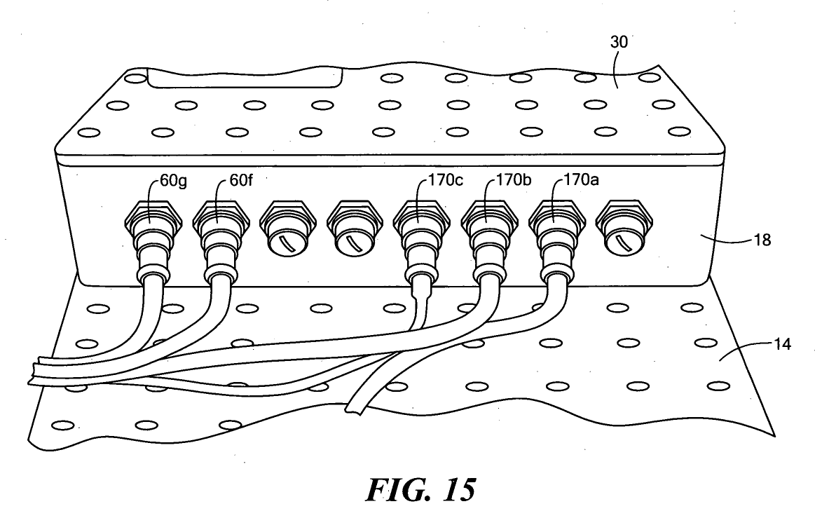

Electronic interface unit 18, FIG. 15 includes connection 170a for the X-ray source, connection 170b for an optional distance sensor (180, FIG. 12), connector 170c for the laser (126, FIG. 12), and USB connector 60f and battery connection 60g, discussed above. Or, a different interface unit may be used and specially configured for the X-ray source.

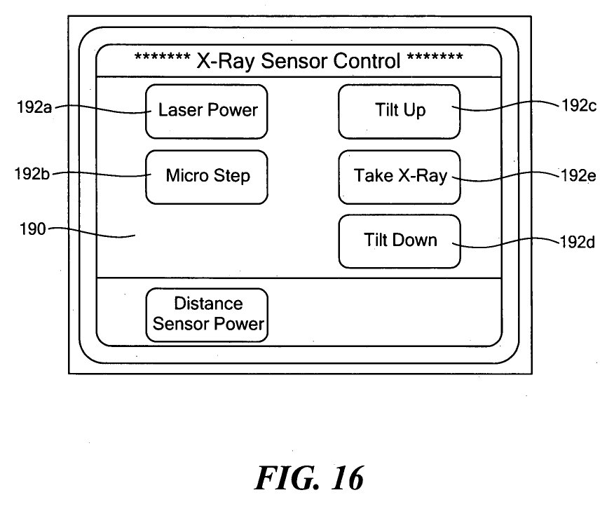

The operator control unit 22, FIG. 16, in one example, includes touch screen 190 which enables the user to power the laser on via button 192a, finely control the movement of the robot platform via button 192b, tilt the X-ray source up via button 192c, tilt the X-ray source down via button 192d, and take an X-ray via button 192e.

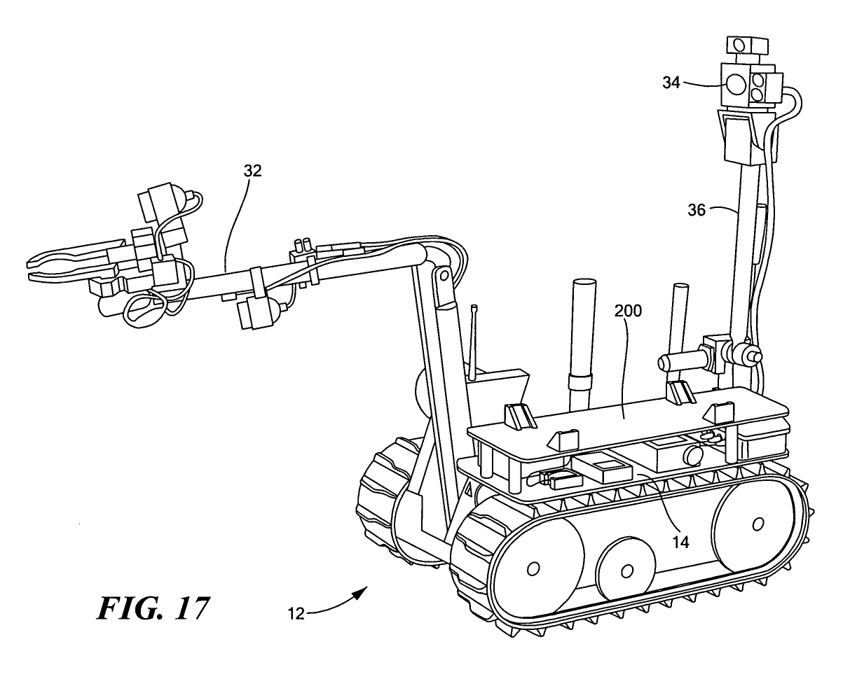

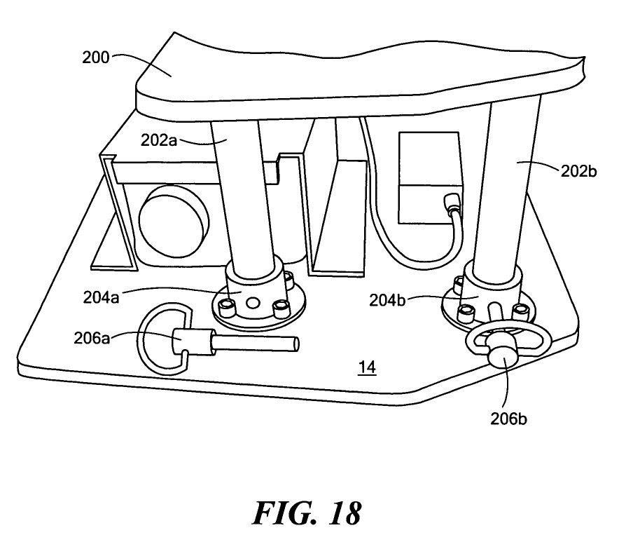

FIGS. 17-19 show optional top rack 200 secured above universal sensor mount tray 14 via posts (typically four) such as posts 202a and 202b, FIG. 18 received in sockets 204a and 204b, respectively, mounted on universal sensor mount tray 14. The posts are releasably retained in the sockets via pins 206a and 206b.

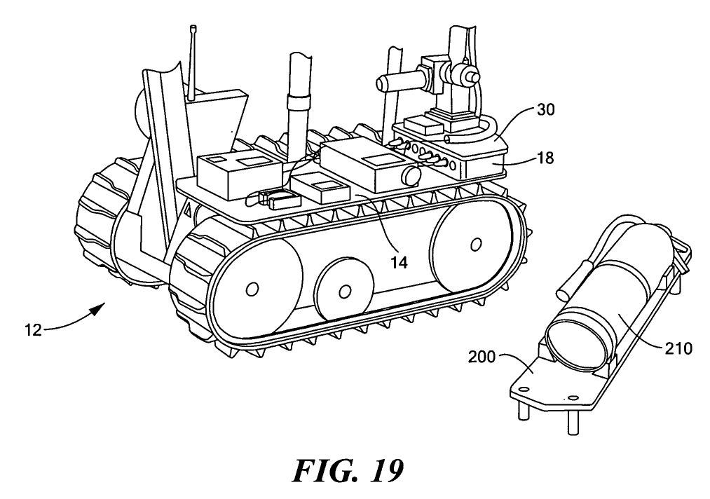

In this way, an item such as fire extinguisher 210, FIG. 19 or other item such as an oxygen tank can be quickly secured to top rack 200 which can be then secured above the sensors on universal sensor rack 14 and brought into an area to be used by hazmat personnel without the need to remove universal sensor rack 14.

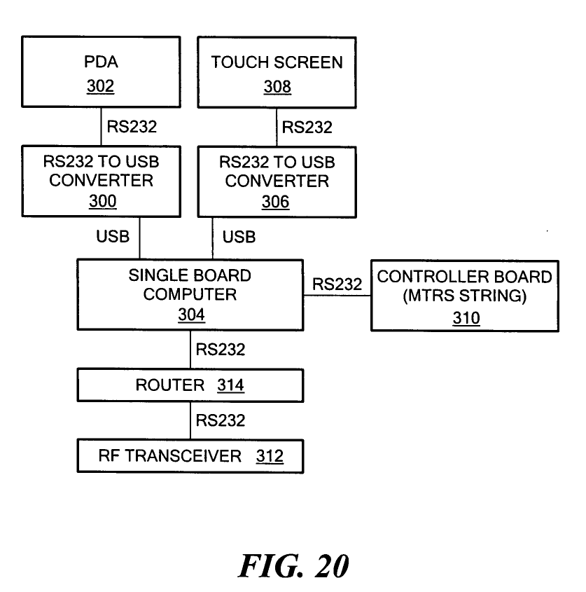

Operator control unit 22, FIG. 5 typically includes converter 300, FIG. 20 for converting an RS 232 interface of PDA 302 to USB signals supplied to and from computer 304. Converter 306 converts the RS 232 interface of touch screen 308 to USB signals also supplied to and from computer 304. Touch screen 308 is used to control X-ray unit 122, FIG. 13 and tilt motor 156, FIG. 14 as shown in FIG. 16. Computer 304, FIG. 20 interfaces with controller board 310 via a RS 232 interface. Communications to and from the robot are via RF transceiver 312 through router 314.

Electronic interface 18, FIG. 3 typically includes X-ray motor 156 (see FIG. 14), controller board 320 and router 322. Controller board 320 controls X-ray motor 156, the activation of the X-ray unit (X-ray shot control 324) and laser 35. Distance sensor 180 and all the sensors (16a-16d, FIG. 3) provide signals to router 322.

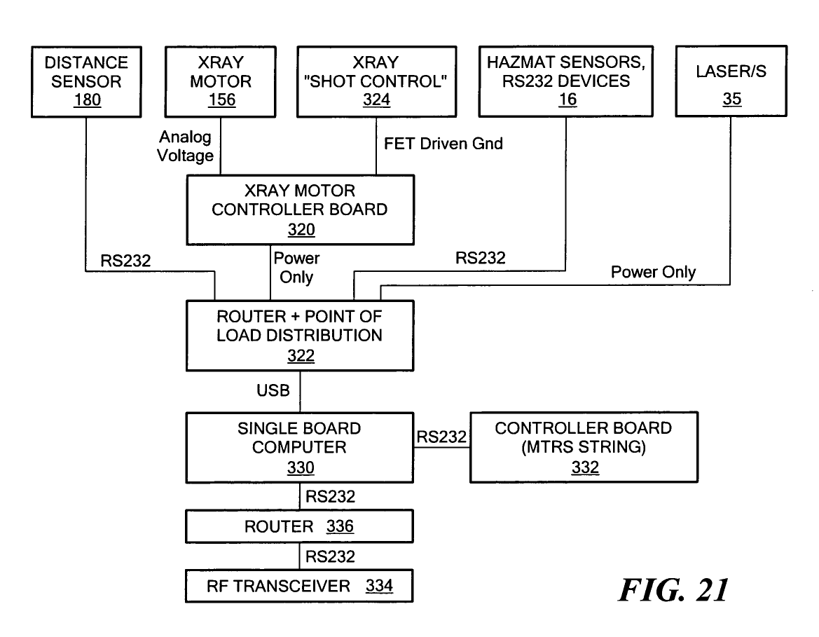

Robot 12, FIG. 2 includes computer 330, FIG. 21 which interfaces with controller board 332. RF transceiver 334 receives and sends signals to the transceiver (312, FIG. 20) of the operator control unit which are processed by computer 330 via router 336. In this way, signals output by any sensor 16, FIG. 21 is displayed on operator control unit 22, FIG. 5 and/or PDA 302, FIG. 20 and X-ray control is effected via touch screen 308 (see FIG. 16). For example, a signal output by a sensor 16, FIG. 21 is routed via router 322 to computer 330 where it is processed for transmission via transceiver 334 to transceiver 312, FIG. 20 of the operator control unit. Computer 304 then processes the signal for display on PDA 302.

The result in any embodiment is an improved hazardous material sensing robot which is more versatile and adaptable. The robot readily accepts output from different types of sensors and from sensors provided by different vendors. The robot is easy to configure, reconfigure, and operate. The hazmat sensors and electronics can be quickly removed so the robot can be used in non-hazmat missions. Also, the hazmat sensors and electronics can be quickly installed so a robot can be used should any mission require hazmat sensors. The quick release universal mounting tray which itself includes an electronic interface unit allows multiple sensors to be mounted thereto and connected to the electronic interface unit in a highly versatile and ergonomic fashion. If needed, an X-ray source can be added. Also, another tray can be provided for carrying either additional sensors and/or items such as fire extinguishers and oxygen tanks.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

What is claimed is:

1. A hazardous materials sensing robot comprising:
a robot platform;
a universal mounting tray on the robot platform for removably mounting thereon a plurality of sensors each having an output;
an electronic interface unit configured to receive the outputs of the sensors;
an operator control unit for remotely operating the robot platform; and
a communication link between the robot platform and the operator control unit for transmitting the sensor outputs to the operator control unit.

2. The robot of claim 1 in which the universal mounting tray includes quick release mounts facilitating removal of the universal mounting tray from the robot platform.

3. The robot of claim 2 further including an interface bracket for each quick release mount.

4. The robot of claim 1 further including a plurality of sensor brackets for mounting the sensors on the universal mounting tray.

5. The robot of claim 1 in which the universal mounting tray includes a broad flat top surface.

6. The robot of claim 5 in which the electronic interface unit is mounted on one end of the broad flat top surface.

7. The robot of claim 6 further including a sensor mounting tray over the electronic interface unit.

8. The robot of claim 1 in which the electronic interface unit includes one or more processors and software that implements the Joint Architecture for Unmanned Systems, operating thereon for accepting the outputs of a variety of different sensors.

9. The robot of claim 1 further including sensors mounted on the universal mounting tray including a chemical sensor, a radiation monitor, a gas detector, an explosives detector, and/or a temperature probe.

10. The robot of claim 1 further including a camera aimable at the universal mounting tray for viewing the output of any sensor mounted thereon not connected to the electronic interface unit.

11. The robot of claim 1 further including a personal data assistant connectable to the operator control unit for displaying the output of the sensors.

12. The robot of claim 1 further including a mount on the universal mounting tray for an X-ray source.

13. The robot of claim 12 in which said mount is configured to pitch up and down.

14. The robot of claim 13 in which said mount is motor driven.

15. The robot of claim 14 in which said motor is controllable by the operator control unit.

16. The robot of claim 12 further including a laser mount for aiming the X-ray source.

17. The robot of claim 12 in which the robot platform further includes an articulating arm including a distal holder.

18. The robot of claim 17 in which the holder includes a bracket pivotably attached to a rod.

19. The robot of claim 12 in which the electronic interface unit includes a connection for the X-ray source.

20. The robot of claim 12 in which the operator control unit includes a touch screen display for controlling the X-ray source.

21. A hazardous materials sensing robot comprising:
a universal mounting tray removably securable to a robot platform via quick release mounts; and
an electronic interface unit configured to receive the outputs of sensors mounted on the universal mounting tray.

22. The robot of claim 21 further including an interface bracket for each quick release mount.

23. The robot of claim 21 further including a plurality of sensor brackets for mounting the sensors on the universal mounting tray.

24. The robot of claim 21 in which the universal mounting tray includes a broad flat top surface.

25. The robot of claim 24 in which the electronic interface unit is mounted on one end of the broad flat top surface.

26. The robot of claim 21 further including a sensor mounting tray over the electronic interface unit.

27. The robot of claim 1 in which the electronic interface unit includes one or more processors and software that implements the Joint Architecture Unmanned Systems operating thereon for accepting the outputs of a variety of different sensors.

28. The robot of claim 21 further including sensors mounted on the universal mounting tray including a chemical sensor, a radiation monitor, a gas detector, an explosives detector, and/or a temperature probe.

29. The robot of claim 21 in which the universal mounting tray includes a camera for viewing the output of any sensor mounted thereon not connected to the electronic interface unit.

30. The robot of claim 21 further including a personal data assistant for displaying the output of the sensors.

31. The robot of claim 21 further including a mount on the universal mounting tray for an X-ray source.

32. The robot of claim 31 in which said mount is configured to pitch up and down.

33. The robot of claim 32 in which said mount is motor driven.

34. The robot of claim 31 further including a laser mount for aiming the X-ray source.

35. The robot of claim 31 further including a holder.

36. The robot of claim 35 in which the holder includes a bracket pivotably attached to a rod.

37. The robot of claim 31 in which the electronic interface unit includes a connection for the X-ray source.

38. A hazardous materials sensing robot comprising:
a robot platform including an articulating arm with a distal holder; and
a mount for an X-ray source for emitting X-rays in the direction of the holder.

39. The robot of claim 38 further including a universal mounting tray on the robot platform, the mount located on the universal mounting tray.

40. The robot of claim 39 further including an electronic interface unit on the universal mounting tray, the X-ray source connected to the electronic interface unit.

41. The robot of claim 38 further including an operator control unit for remotely operating the robot platform and the X-ray source.

42. The robot of claim 39 in which said mount is configured to pitch up and down.

43. The robot of claim 38 in which said mount is motor driven.

44. The robot of claim 41 in which said motor is controllable by the operator control unit.

45. The robot of claim 38 further including a laser mount for aiming the X-ray source.

46. The robot of claim 41 in which the operator control unit includes a touch screen display for controlling the X-ray source.

47. A hazardous materials sensing system for a robot, the system comprising:
a universal mounting tray for mounting on a robot platform and for removably mounting thereon a plurality of sensors each having an output; and
an electronic interface unit on the universal mounting tray configured to receive and process the outputs of the sensors.

48. The system of claim 47 further including a second tray over the electronic interface unit.

49. The system of claim 47 further including a third tray releasably fixed over the universal mounting tray.
